(19) United States
(12) Patent Application Publication
(10) Pub. No.: US 2007/0269525 A1
(43) Pub. Date: Nov. 22, 2007

(54) O/W Gel Compositions Comprising Wax

(76) Inventors: Helga Gondek, Duesseldorf (DE); Ulrich Issberner, Rommerskirchen (DE); Catherine Weichold, Aachen (DE)

Correspondence Address: COGNIS CORPORATION;PATENT DEPARTMENT, 300 BROOKSIDE AVENUE, AMBLER, PA, 19002, US

(21) Appl. No.: 10/599,683

(22) PCT Filed: Mar. 26, 2005
(86) PCT No.: PCT/EP05/03235

§ 371 Date: Jul. 25, 2007

(30) Foreign Application Priority Data
Apr. 5, 2004 (DE) 10 2004 017 223.4

(51) Int. Cl. A61K 31/74; A61K 9/50; A61P 43/00

(52) U.S. Cl. 424/502; 424/78.08

(57) ABSTRACT

An oil in water gel composition including (a) from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof; (b) from about 0.1% to about 10% by weight of at least one wax component with a melting point of at least 30° C.; (c) from about 1% to about 30% by weight of an oil component which is liquid at 25° C.; and (d) from about 60% to about 95% by weight of water, with the proviso that the composition is free from anionic and cationic emulsifiers and surfactants is provided. Processes for producing the compositions and incorporation of the compositions into a cosmetic formulation are also provided.

### RELATED APPLICATIONS

This application is a national phase filing under 35 U.S.C. § 371 and claims priority to International Application No. PCT/EP2005/003235 which has an International filing date of Mar. 26, 2005, and which designated the United States of America and which claims priority to German Application No. 10 2004 017 223.4, filed Apr. 5, 2004, the entire disclosures of which are hereby incorporated herein by reference.

### FIELD OF THE INVENTION

This invention relates generally to oil in water (O/W) gel formulations, and more specifically, to O/W gel formulations including a polymeric gel former, a process for their production, and their use in a skin care formulation.

### BACKGROUND INFORMATION

Polyacrylate-based gel formulations have long been known to the expert. Many of these gel formulations are surfactant-containing preparations which are used for personal hygiene. Thus, WO 96/17591 and WO 96/17592 describe surfactant-containing skin-cleansing formulations which are stabilized by polymeric gel formers. Although gels impart a sensorially very light and pleasantly cooling sensation, they are not normally used for the formulation of skin-care preparations because their care effects are poor. Polyacrylate-based gel formulations in particular do not have lasting care effects. In addition, gel formulations are salt-sensitive so that, when applied to the skin, they often break through the presence of salts.

The problem addressed by the present invention was to provide gel formulations which would have a pleasant, light sensory profile and good care effects and which would not break when applied to the skin. Another problem addressed by the invention was to provide irritation-free formulations.

It has now surprisingly been found that these properties can be achieved by the formulation of o/w gels which are free from the usual cationic and anionic surfactants and emulsifiers and which contain a combination of certain gel formers and waxes and oils.

### SUMMARY OF THE INVENTION

Briefly described, according to an aspect of the invention, an oil in water gel composition includes (a) from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof; (b) from about 0.1% to about 10% by weight of at least one wax component with a melting point of at least 30° C.; (c) from about 1% to about 30% by weight of an oil component which is liquid at 25° C.; and (d) from about 60% to about 95% by weight of water, with the proviso that the composition is free from anionic and cationic emulsifiers and surfactants.

In another aspect of the invention, a process for the production of an oil in water gel composition includes: (a) providing an oil phase comprising from about 0.1% to about 10% by weight of at least one wax component with a melting point of at least 30° C. and from about 1% to about 30% by weight of an oil component which is liquid at 25° C.; (b) dispersing in the oil phase from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof to form a dispersion; (c) emulsifying the dispersion in water to form a composition, with the proviso that the composition is free from anionic and cationic emulsifiers and surfactants.

In another aspect of the invention, a process for the production of an oil in water gel composition includes: (a) combining from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof with water to form a swollen polymeric gel former; and (b) combining the swollen polymeric gel former with a liquid oil phase comprising from about 0.1% to about 10% by weight of at least one wax component with a melting point of at least 30° C. and from about 1% to about 30% by weight of an oil component which is liquid at 25° C. to form a composition, with the proviso that the composition is free from anionic and cationic emulsifiers and surfactants.

In yet another aspect of the invention, a process for the production of an oil in water gel composition includes: (a) combining from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof with at least one low molecular weight polyol with a molecular weight of <1,000 dalton to form a swollen polymeric gel former; (b) processing the swollen polymeric gel former with water and a liquid oil phase comprising from about 0.1% to about 10% by weight of at least one wax component with a melting point of at least 30° C. and from about 1% to about 30% by weight of an oil component which is liquid at 25° C. to form a composition, with the proviso that the composition is free from anionic and cationic emulsifiers and surfactants.

In yet another aspect of the invention, the above-described composition may be incorporated into a body care formulation.

### DETAILED DESCRIPTION OF THE INVENTION

Accordingly, the present invention relates to o/w gel compositions containing (a) 0.05 to 5% by weight of at least one polymeric gel former selected from the group of homopolymers or copolymers of acrylic acid and/or acrylamide and derivatives thereof, (b) 0.1 to 10% by weight of at least one wax component with a melting point of at least 30° C., (c) 1 to 30% by weight of an oil component liquid at 25° C. and (d) 60 to 95% by weight of water, with the proviso that the composition does not contain any anionic or cationic emulsifiers/surfactants.

Compositions of this type are far more stable to the salt content on the skin and, after application, leave the skin feeling smooth and soft with very good care properties. They are easy to apply and spread, are readily absorbed by the skin and leave the skin feeling velvety rather than oily or greasy. By virtue of the absence of relatively large quantities of emulsifiers and surfactants, the compositions have a low irritation potential.

The compositions according to the invention do not contain any additional anionic or cationic surfactants/emulsifiers. Preferably, they also contain no nonionic ethoxylated surfactants/emulsifiers or only very small quantities thereof which can emanate from the raw materials used. This quantity is normally below 0.5% by weight, preferably below 0.3% by weight and more particularly below 0.1% by weight, based on the composition as a whole.

The o/w gel compositions according to the invention preferably have a viscosity at 20° C. of 50,000 to 500,000 mPa·s, as measured with a Brookfield RVF viscosimeter, spindle TE with Helipath, at 4 r.p.m.

Gel Formers

The gel formers are selected from the group of homopolymers or copolymers of acrylic acid and/or acrylamide and derivatives thereof or from a mixture of these substances. These include commercially available substances such as, for example, Aristoflex® AVC, INCI: Ammonium Acryloyldimethyltaurate/VP Copolymer; Aristoflex® AVC-1, INCI: Ammonium Acryloyldimethyltaurate/Vinyl Formamide Copolymer; Aristoflex® HMB, INCI: Ammonium Acryloyldimethyltaurate/Beheneth-25-Methacrylate Copolmer; Pemulen® TR-1, INCI: Acrylates/C10-30 Alkyl Acrylate Crosspolymer; Pemulen® TR-2: Acrylates/C10-30 Alkyl Acrylate Crosspolymer; Carbopol® 980, INCI: Carbomer (for example homopolymers of acrylic acid crosslinked with an allyl ether of pentaerythritol, an allyl ether of sucrose or an allyl ether of propylene); Carbopol® ETD 2020, INCI: Acrylates/C10-30 Alkyl Acrylate Crosspolymer; Carbopol® Ultrez 10, INCI: Carbomer.

The polymers may be crosslinked or uncrosslinked. Crosslinked polymers are preferably used. According to the invention, polyacrylates and polyacrylamides are preferred. Sodium polyacrylates are particularly preferred. According to the invention, a most particularly preferred polymer is commercially available under the name of Cosmedia® SP. According to the invention, the polymers are used in quantities of 0.05 to 5% by weight, based on the composition as a whole. Quantities of 0.1 to 4% by weight are preferred, quantities of 0.5 to 3% by weight are particularly preferred and quantities of 0.5 to 2% by weight—based on the composition as a whole—are most particularly preferred.

Wax Components

Waxes are normally understood to be any natural or synthetic substances and mixtures having the following properties: they have a solid to brittle hard consistency, are coarsely to finely crystalline, transparent to opaque and melt above 30° C. without decomposing. Even slightly above their melting point, they are low in viscosity and non-stringing and are very temperature-dependent in their consistency and solubility. A wax component or a mixture of wax components which melt(s) at 30° C. or higher may be used in accordance with the invention. They are present in the compositions according to the invention in a total quantity of 0.1 to 10% by weight. In a preferred embodiment of the invention, the content of wax component is from 0.2 to 5% by weight, based on the composition as a whole. Quantities of 0.5 to 4% by weight—based on the composition as a whole—are preferred and quantities of 0.5 to 2% by weight particularly preferred. A content of 0.5 to 1.5% by weight of the wax component(s), based on the composition as a whole, is most particularly preferred because the overall sensory profile is optimal in that range. Another preferred embodiment of the o/w gel composition is characterized in that the wax component (b) has a melting point of 40° C. to 80° C. and preferably in the range from 40° C. to 60° C. because the best sensory effects are obtained in that range.

According to the invention, fats and fat-like substances with a wax-like consistency may also be used as waxes, providing they have the required melting point. These include inter alia fats (triglycerides), mono- and diglycerides, natural and synthetic waxes, fatty and wax alcohols, fatty acids, esters of fatty alcohols and fatty acids and also fatty acid amides or mixtures of these substances.

Fats in the context of the invention are understood to be triacylglycerols, i.e. the triple esters of fatty acids with glycerol. They preferably contain saturated, unbranched and unsubstituted fatty acid components. They may also be mixed esters, i.e. triple esters of glycerol with various fatty acids. So-called hardened fats and oils obtained by partial hydrogenation may be used in accordance with the invention and are particularly suitable as consistency factors. Vegetable hardened fats and oils, for example hardened castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat, are preferred.

Suitable fats are inter alia the triple esters of glycerol with C₁₂₋₆₀ fatty acids and in particular C₁₂₋₃₆ fatty acids. These include hydrogenated castor oil, a triple ester of glycerol and a hydroxystearic acid which is marketed, for example, under the name of Cutina® HR. Gycerol tristearate, glycerol tribehenate (for example Syncrowax® HRC), glycerol tripalmitate or the triglyceride mixtures known under the name of Syncrowax® HGLC, are also suitable providing the melting point of the wax component or the mixture as a whole is 30° C. or higher.

According to the invention, suitable wax components are, in particular, mono- and diglycerides and mixtures of these partial glycerides. Glyceride mixtures suitable for use in accordance with the invention include the products Novata® AB and Novata® B (mixture of C₁₂₋₁₈ mono-, di- and triglycerides) and Cutina® MD or Cutina® GMS (glyceryl stearate) marketed by Cognis Deutschland GmbH & Co. KG.

Mixed esters and mixtures of mono-, di- and triglycerides are particularly suitable for the purposes of the invention because they have a relatively low tendency towards crystallization and thus improve the performance of the composition according to the invention.

Fatty alcohols suitable for use in accordance with the invention include C₁₂₋₅₀ fatty alcohols and, more particularly, C₁₂₋₂₄ fatty alcohols obtained from natural fats, oils and waxes such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. According to the invention, saturated, unbranched fatty alcohols are preferred. However, unsaturated, branched or unbranched fatty alcohols may also be used as wax component for the purposes of the invention providing they have the required melting point. Other suitable fatty alcohols are the fatty alcohol cuts obtained in the reduction of naturally occurring fats and oils such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut oil. However, synthetic alcohols, for example the linear, even-numbered fatty alcohols from Ziegler's synthesis (Alfols®) or the partly branched alcohols from the oxosynthesis (Dobanols®) may also be used. The C₁₄₋₂₂ fatty alcohols marketed, for example, by Cognis Deutschland GmbH under the name of Lanette® 16 (C₁₆ alcohol), Lanette® 14 (C₁₄ alcohol), Lanette® O (C_{16/18} alcohol) and Lanette® 22 (C_{18/22} alcohol) are particularly suitable for the purposes of the invention. Fatty alcohols give the compositions a drier feeling on the skin than triglycerides and are therefore preferred to triglycerides.

C₁₄₋₄₀ fatty acids or mixtures thereof may also be used as wax components. These include, for example, myristic, pentadecanoic, palmitic, margaric, stearic, nonadecanoic, arachic, behenic, lignoceric, cerotic, melissic, erucic and elaeostearic acid and substituted fatty acids such as, for example, 12-hydroxystearic acid, and the amides or monoethanolamides of the fatty acids. This list is meant to be purely exemplary without any limiting character.

Waxes suitable for use in accordance with the present invention are, for example, natural vegetable waxes, such as candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. According to the invention, it can be of advantage to use hydrogenated or hardened waxes. Natural waxes usable in accordance with the invention also include the mineral waxes, such as ceresine and ozocerite for example, or the petrochemical waxes, for example petrolatum, paraffin waxes and microwaxes. Other suitable wax components are chemically modified waxes, more particularly the hard waxes such as, for example, montan ester waxes, sasol waxes and hydrogenated jojoba waxes. Synthetic waxes usable in accordance with the invention include, for example, wax-like polyalkylene waxes and polyethylene glycol waxes. Vegetable waxes are preferred for the purposes of the invention.

The wax component may also be selected from the group of wax esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids. Examples of such esters are the C₁₆₋₄₀ alkyl stearates, C₂₀₋₄₀ alkyl stearates (for example Kesterwachs® K82H), C₂₀₋₄₀ dialkyl esters of dimer acids, C₁₈₋₃₈ alkyl hydroxystearoyl stearates or C₂₀₋₄₀ alkyl erucates, esters of alkyl carboxylic acids with pentaerythritol, dipentaerythritol and tripentaerythritol or a mixture of the esters of the pentaerythritol oligomers. C₃₀₋₅₀ alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetearyl behenate and behenyl behenate may also be used.

In a preferred embodiment of the invention, the wax component (b) with a melting point of at least 30° C. is selected from the group of pentaerythritol esters, dipentaerythritol esters, tripentaerythritol esters, C₁₄₋₂₂ fatty alcohols, more particularly C₁₆₋₁₈ fatty alcohols, and partial glycerides or mixtures of these substances. In another preferred embodiment, the esters are esters of saturated or unsaturated and/or branched or unbranched C₆₋₂₂ fatty acids—preferably C₁₄₋₂₂ fatty acids and more particularly C₁₆₋₂₂ fatty acids—of pentaerythritol, dipentaerythritol, tripentaerythritol or mixtures of these esters, less than 0.3% by weight C₁₇ fatty acid esters being present.

Particularly preferred esters are esters of pentaerythritol which are obtained by reaction of pentaerythritol with a fatty acid mixture containing 40 to 50% by weight C₁₆ fatty acid(s) and 45 to 55% by weight C₁₈ fatty acid(s) and which have a percentage content of (a) 5 to 35% by weight monoesters, (b) 20 to 50% by weight diesters and (c) 25 to 50% by weight triesters and optionally tetraesters. Esters of pentaerythritol which are obtained by reaction of pentaerythritol with a fatty acid mixture containing 40 to 50% by weight C₁₆ fatty acid(s) and 45 to 55% by weight C₁₈ fatty acid(s) and which have the following ester distribution: (a) 12 to 19% by weight monoesters, (b) 25 to 35% by weight diesters and (c) 30 to 40% by weight triesters and 6 to 11% by weight tetraesters.

Oil Components

The o/w gels according to the invention contain 1 to 30% by weight, based on the composition as a whole, of an oil component liquid at 25° C. or a mixture of such oil components. The oil component(s) is/are present in a total quantity of 3 to 20% by weight, preferably in a quantity of 5 to 15% by weight and more particularly in a quantity of 7 to 12% by weight. Suitable oil components are, for example, the classes of compounds mentioned in the following, providing they are liquid at 25° C. These include inter alia Guerbet alcohols based on fatty alcohols containing 6 to 18 and preferably 8 to 10 carbon atoms, esters of linear or branched, saturated or unsaturated C₆₋₂₂ fatty acids with linear or branched, saturated or unsaturated C₆₋₂₂ fatty alcohols, more particularly 2-ethyl hexanol. The following are mentioned by way of example: hexyl laurate, myristyl isostearate, myristyl oleate, cetyl isostearate, cetyl oleate, stearyl isostearate, stearyl oleate, isostearyl myristate, isostearyl palmitate, isostearyl stearate, isostearyl isostearate, isostearyl oleate, oleyl myristate, oleyl isostearate, oleyl oleate, oleyl erucate, erucyl isostearate, erucyl oleate, cococaprylate/caprate. Other suitable esters are, for example, esters of C₁₈₋₃₈ alkylhydroxycarboxylic acids with linear or branched, saturated or unsaturated C₆₋₂₂ fatty alcohols, esters of linear and/or branched, saturated and/or unsaturated fatty acids with polyhydric alcohols (for example propylene glycol, dimer diol or trimer triol) and/or Guerbet alcohols, triglycerides or triglyceride mixtures, mono-, di- and triglyceride mixtures, esters of C₆₋₂₂ fatty alcohols and/or Guerbet alcohols with aromatic carboxylic acids, more particularly benzoic acid, esters of C₂₋₁₂ dicarboxylic acids with linear or branched, saturated or unsaturated alcohols containing 1 to 22 carbon atoms or polyols containing 2 to 10 carbon atoms and 2 to 6 hydroxyl groups, vegetable oils, branched primary alcohols, substituted cyclohexanes, linear dialkyl carbonates, Guerbet carbonates based on fatty alcohols containing 6 to 18 and preferably 8 to 10 carbon atoms, esters of benzoic acid with linear and/or branched C₆₋₂₂ alcohols (for example Cetiol® AB), linear or branched, symmetrical or nonsymmetrical dialkyl ethers containing 6 to 22 carbon atoms per alkyl group such as, for example, Di-n-octyl Ether (Cetiol® OE) or ring opening products of epoxidized fatty acid esters with polyols, hydrocarbons, such as paraffin or mineral oils, silicone oils and oligo- or poly-α-olefins. olefins.

According to the invention, dialkyl carbonates and triglycerides or mixtures thereof are preferred oil components. The dialkyl carbonates may be symmetrical or asymmetrical, branched or unbranched, saturated or unsaturated and may be produced by transesterification reactions known from the prior art. According to the invention, dialkyl carbonates with alkyl chains containing 6 to 24 carbon atoms, more particularly di-n-octyl carbonate or di-(2-ethylhexyl) carbonate, or mixtures thereof are particularly suitable. Of these, di-n-octyl carbonate is preferred.

The hydrocarbons suitable for use in accordance with the invention have a chain length of 8 to 40 carbon atoms. They may be branched or unbranched, saturated or unsaturated. Of these, branched, saturated C₈₋₄₀ alkanes are preferred. Both pure substances and mixtures may be used. The mixtures are normally mixtures of different isomeric compounds. Compositions containing C₁₀₋₃₀, preferably C₁₂₋₂₀ and more particularly C₁₆₋₂₀ alkanes are particularly suitable and, of these, a mixture of alkanes containing at least 10% by weight branched alkanes, based on the total quantity of alkanes, is particularly preferred. The alkanes are preferably branched, saturated alkanes. Mixtures of alkanes containing more than 1% by weight 5,8-diethyl dodecane and/or more than 1% by weight didecene are particularly suitable.

A preferred embodiment of the o/w gel composition according to the invention contains (a) 0.05 to 5% by weight of at least one polyacrylate, (b) 0.1 to 10% by weight of at least one ester of pentaerythritol and/or dipentaerythritol, (c) 1 to 30% by weight of at least one oil component and (d) 60 to 95% by weight water. A particularly preferred o/w gel composition contains (a) 0.05 to 5% by weight of at least one sodium polyacrylate, (b) 0.1 to 10% by weight of at least one pentaerythrityl partial ester or dipentaerythrityl partial ester based on stearic and/or palmitic acid, (c) 1 to 30% by weight of at least one oil component selected from fatty acid esters or dialkyl carbonates liquid at 25° C. or a mixture thereof and (d) 60 to 95% by weight water.

Another preferred embodiment is an o/w gel composition containing (a) 0.5 to 2% by weight of at least one sodium polyacrylate, (b) 0.5 to 2.0% by weight of at least one wax component with a melting point of at 30° C. selected from the group of pentaerythrityl esters or dipentaerythrityl esters based on stearic and palmitic acid, C₁₆₋₁₈ fatty alcohols and/or C₁₆₋₁₈ partial glycerides, (c) 7 to 12% by weight of at least one oil component selected from fatty acid esters, triglycerides, dialkyl carbonates, hydrocarbons, dialkyl ethers liquid at 25° C. or mixtures thereof and (d) 60 to 95% by weight water.

The present invention also relates to a process for the production of the o/w gel composition according to the invention in which either a) the gel former or a mixture of the gel formers (a) is dispersed in the liquid oil phase containing the wax and oil components and the resulting dispersion is subsequently emulsified with the aqueous phase or b) the gel former or a mixture of the gel formers is swollen in the aqueous phase and the whole is mixed with the liquid oil phase or c) the gel former or a mixture of the gel formers is swollen in a low molecular weight polyol or polyol mixture with a molecular weight of <1,000 dalton and the whole is processed with the aqueous phase and with the liquid oil phase.

The present invention also relates to the use of the o/w gel composition according to the invention for body care. The present invention also relates to the use of waxes for improving the salt tolerance of gel compositions containing the gel formers claimed in claim 1.

Other Optional Auxiliaries and Additives

Depending on their intended application, the cosmetic formulations contain a number of other auxiliaries and additives such as, for example, other thickeners, superfatting agents, stabilizers, polymers, lecithins, phospholipids, biogenic agents, UV protection factors, antioxidants, deodorants, film formers, swelling agents, insect repellents, hydrotropes, solubilizers, preservatives. perfume oils, dyes, etc. which are listed by way of example in the following. The quantities in which the particular additives are used is determined by the intended use.

Other suitable thickeners are, for example, Aerosil®) types (hydrophilic silicas), polysaccharides, more especially xanthan gum, guar-guar, agar-agar, alginates and tyloses, carboxymethyl cellulose and hydroxyethyl and hydroxypropyl cellulose, polyvinyl alcohol, polyvinyl pyrrolidone and bentonites such as, for example, Bentone® GeIVS-5PC (Rheox).

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. Typical UV-A filters are, in particular, derivatives of benzoyl methane. The UV-A and UV-B filters may of course also be used in the form of mixtures, for example combinations of the derivatives of benzoyl methane, for example 4-tert.butyl4′-methoxydibenzoylmethane (Parsol® 1789) and 2-cyano-3,3-phenylcinnamic acid-2-ethyl hexyl ester (Octocrylene), and esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are often combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide.

Besides the two groups of primary sun protection factors mentioned above, secondary sun protection factors of the antioxidant type may also be used. Secondary sun protection factors of the antioxidant type interrupt the photochemical reaction chain which is initiated-when UV rays penetrate into the skin.

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Deodorizing components counteract, mask or eliminate body odors. Body odors are formed through the action of skin bacteria on apocrine perspiration which results in the formation of unpleasant-smelling degradation products. Accordingly, suitable deodorizing components are inter alia germ inhibitors, enzyme inhibitors, odor absorbers or odor maskers.

Suitable insect repellents are, for example, N,N-diethyl-m-toluamide, pentane-1,2-diol or 3-(N-n-butyl-N-acetylamino)-propionic acid ethyl ester), which is marketed as Insect Repellent® 3535 by Merck KGaA, and Butylacetyl-aminopropionate.

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

In addition, hydrotropes, such as for example ethanol, isopropyl alcohol or polyols, may be used to improve flow behavior. Suitable polyols preferably contain 2 to 15 carbon atoms and at least two hydroxyl groups.

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Suitable perfume oils are mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, and synthetic perfume compounds of the ester, ether, aldehyde, ketone, alcohol and hydrocarbon type, are also suitable.

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). These dyes are normally used in concentrations of 0.001 to 0.1% by weight, based on the mixture as a whole.

### EXAMPLES

The quantities shown in the following Table represent % by weight of the commercially available substance in the composition as a whole.

| Ingredient | | | | | | | | | |
|---|---|---|---|---|---|---|---|---|---|
| (INCl/commercial name) | C1 | 1 | 2 | 3 | 4 | 5 | 6 | 7 | 8 |
| Sodium polyacrylate | 1.0 | 1.0 | 1.0 | 1.0 | 1.0 | 1.0 | 1.0 | 1.0 | 1.0 |
| (Cosmedia ® SP) | | | | | | | | | |
| Pentaerythritol Distearate | | 1.0 | 2.0 | | | 1.0 | 1.0 | | |
| Pentaerythritol Distearate/ | | | | 1.0 | | | | 1.0 | 1.0 |
| Dipentaerythrityl stearyl | | | | | | | | | |
| ester | | | | | | | | | |
| Cetearyl Alcohol | | | | 1.0 | | | | | |
| (Lanette ® O) | | | | | | | | | |
| Glyceryl Stearate | | | | | 1.0 | | | | |
| (Cutina ® GMS-V) | | | | | | | | | |
| Dicaprylyl Carbonate | 5.0 | 5.0 | 5.0 | 5.0 | 5.0 | 5.0 | | 5.0 | 4.5 |
| (Cetiol ® CC) | | | | | | | | | |
| Coco-Caprylate/Caprate | 5.0 | 5.0 | 5.0 | 5.0 | 5.0 | 5.0 | | 5.0 | 4.5 |
| (Cutina ® LC) | | | | | | | | | |
| Cyclomethicone | | | | | | | 10.0 | | |
| (Dow Corning ® 245) | | | | | | | | | |
| Dimer Distearyltricarbonate | | | | | | | | | 1.0 |
| Cosmeida ® DC) | | | | | | | | | |
| Ethanol | | | | | | 15.0 | | | |
| Water | to | to | to | to | to | to | to | to | to |
| | 100 | 100 | 100 | 100 | 100 | 100 | 100 | 100 | 100 |
| Evaluation | | | | | | | | | |
| Smoothness | − | ++ | ++ | + | + | ++ | ++ | ++ | ++ |
| Softness | − | ++ | + | + | + | + | ++ | ++ | ++ |
| Acceptance | − | ++ | + | + | + | ++ | ++ | ++ | ++ |

= excellent;
+ = very good;
0 = good;
− = average;
— = unsatisfactory

Test group: 10 experienced and trained volunteers

10 μl of the above compositions brought beforehand to 20° C. were applied by micropipette to the hair-free side of the forearm of the volunteers and rubbed in with the fingers of the hands of the contralateral side. The sensory profile was evaluated during and after absorption.

The sensory test was conducted on 10 volunteers, as described in the book “Cosmetic Lipids and the Skin Barrier” (Marcel Dekker, New York, 2002; Ed.: Thomas Förster, pp. 319-352).

What is claimed is:

1. An oil in water gel composition, comprising:
(a) from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof;
(b) from about 0.1% to about 10% by weight of at least one wax component with a melting point of at least 30° C.;
(c) from about 1% to about 30% by weight of an oil component which is liquid at 25° C.; and
(d) from about 60% to about 95% by weight of water,
with the proviso that the composition is free from anionic and cationic emulsifiers and surfactants.

2. The oil in water gel composition according to claim 1, wherein component (b) is present in an amount of from about 0.2% to about 5% by weight, based on the weight of the composition.

3. The oil in water gel composition according to claim 1, wherein component (b) is selected from the group consisting of pentaerythritol esters, dipentaerythritol esters, tripentaerythritol esters, C₁₂₋₁₄ fatty alcohols, C₁₆₋₁₈ fatty alcohols, partial glycerides, and mixtures thereof.

4. The oil in water gel composition according to claim 1, wherein component (b) is a C₁₆₋₁₈ fatty alcohol.

5. The oil in water gel composition according to claim 1, wherein component (b) has a melting point of 40° C. to 80° C.

6. The oil in water gel composition according to claim 1, wherein component (b) is an ester selected from the group consisting of pentaerythritol esters, pentaerythritol oligomers thereof, and mixtures thereof, wherein the ester is esterified with saturated or unsaturated and/or branched or unbranched C₆₋₂₂ fatty acids, and wherein the ester contains less than 0.3% by weight of C₁₇ fatty acid esters.

7. The oil in water gel composition according to claim 1, wherein component (b) is a pentaerythritol ester obtained by reaction of pentaerythritol with a fatty acid mixture containing 40% to 50% by weight C₁₆ fatty acid(s) and 45% to 55% by weight C₁₈ fatty acid(s) and which have a percentage content of (a) 5% to 35% by weight monoesters, (b) 20% to 50% by weight diesters and (c) 25% to 50% by weight triesters.

8. The oil in water gel composition according to claim 7, wherein the percentage content further comprises (d) 6% to 11% by weight tetraesters.

9. The oil in water gel composition according to claim 1, wherein component (a) comprises at least one polyacrylate and component (b) comprises at least one ester selected from the group consisting of pentaerythritol, dipentaerythritol, and mixtures thereof.

10. The oil in water gel composition according to claim 1, wherein component (a) comprises at least one sodium polyacrylate, component (b) comprises at least one pentaerythritol partial ester or at least one dipentaerythritol partial ester, wherein the ester is based on stearic acid, palmitic acid, or mixtures thereof, and component (c) comprises at least one oil component selected from fatty acid esters, dialkyl carbonates, or mixtures thereof.

11. The oil in water gel composition according to claim 1, wherein the composition has a viscosity at 20° C. of 50,000 to 500,000 mPas, as measured with a Brookfield RVF viscosimeter, spindle TE with Helipath, at 4 r.p.m.

12. A process for the production of an oil in water gel composition comprising the steps of:
(a) providing an oil phase comprising from about 0.1% to about 10% by weight of at least one wax component with a melting point of at least 30° C. and from about 1% to about 30% by weight of an oil component which is liquid at 25° C.;
(b) dispersing in the oil phase from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof to form a dispersion;
(c) emulsifying the dispersion in water to form a composition, with the proviso that the composition is free from anionic and cationic emulsifiers and surfactants.

13. A process for the production of an oil in water gel composition comprising the steps of:
(a) combining from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof with water to form a swollen polymeric gel former; and
(b) combining the swollen polymeric gel former with a liquid oil phase comprising from about 0.1% to about 10% by weight of at least one wax component with a melting point of at least 30° C. and from about 1% to about 30% by weight of an oil component which is liquid at 25° C. to form a composition, with the proviso that the composition is free from anionic and cationic emulsifiers and surfactants.

14. A process for the production of an oil in water gel composition comprising the steps of:
(a) combining from about 0.05% to about 5% by weight of at least one polymeric gel former selected from the group consisting of acrylic acid, acrylamide, homopolymers and copolymers of acrylic acid, homopolymers and copolymers of acrylamide, derivatives thereof, and mixtures thereof with at least one low molecular weight polyol with a molecular weight of <1,000 dalton to form a swollen polymeric gel former;
(b) processing the swollen polymeric gel former with water and a liquid oil phase comprising from about 0.1% to about 10% by weight of at least one wax component with a melting point of at least 30° C. and from about 1% to about 30% by weight of an oil component which is liquid at 25° C. to form a composition, with the proviso that the composition is free from anionic and cationic emulsifiers and surfactants.

15. The composition according to claim 1, incorporated into a body care formulation.
